(19) United States
(12) Patent Application Publication
(10) Pub. No.: US 2020/0405632 A1
(43) Pub. Date: Dec. 31, 2020

(54) TRANSCERVICAL TREATMENT OF GYNECOLOGICAL MALIGNANCIES

(71) Applicant: Covidien LP, Mansfield, MA (US)

(72) Inventors: Nikolai D. Begg, Wellesley, MA (US); Chad A. Pickering, Woburn, MA (US); Jordan A. Whisler, Brookline, MA (US); Rebecca D. White, Kennett Square, PA (US)

(21) Appl. No.: 16/905,121

(22) Filed: Jun. 18, 2020

Related U.S. Application Data
(60) Provisional application No. 62/868,207, filed on Jun. 28, 2019.

(51) Int. Cl. A61K 9/00; A61K 45/00; A61K 9/16; A61B 17/42

(52) U.S. Cl. CPC A61K 9/0034; A61K 45/00; A61B 2017/4225; A61B 17/42; A61K 9/1652

(57) ABSTRACT

A spheroidal implant system includes a plurality of spheroidal implants configured to be positioned together within a uterus of a patient. Each spheroidal implant includes a bioactive agent that is selectively releasable into the uterus to enable the spheroidal implants to cooperate together to simultaneously treat a gynecological malignancy of the patient.

### CROSS REFERENCE TO RELATED APPLICATIONS

This application claims the benefit of U.S. Provisional Application Ser. No. 62/868,207, filed Jun. 28, 2019, the entire contents of which are incorporated by reference herein.

### TECHNICAL FIELD

This disclosure relates generally to treatment of gynecological malignancies, and in particular, to transcervical treatment of gynecological malignancies such as endometrial cancer and hyperplasia.

### BACKGROUND

Endometrial cancer is the most common gynecologic malignancy. It is the fourth most common cancer in women in the United States after breast, lung, and colorectal cancers. Endometrial hyperplasia is characterized by a thickening of the endometrium that is more than the typical pre- and post-menstrual buildup of endometrial tissue. Low- to medium-risk endometrial hyperplasia can be treated with nonsurgical options. The mainstay of treatment for endometrial cancer and atypical endometrial hyperplasia is a total hysterectomy. Radiation and chemotherapy can also play a role in treatment.

### SUMMARY

According to one aspect, this disclosure is directed to a spheroidal implant system. The spheroidal implant system includes a plurality of spheroidal implants configured to be positioned together within a uterus of a patient. Each spheroidal implant includes a bioactive agent that is selectively releasable into the uterus to enable the spheroidal implants to cooperate together to simultaneously treat a gynecological malignancy of the patient.

In some embodiments, the bioactive agent may include a chemotherapeutic agent.

In certain embodiments, the bioactive agent may include a hormonal agent.

In embodiments, each of the plurality of spheroidal implants may include a resorbable material. The resorbable material may include carboxy-methyl cellulose. The resorbable material may be configured to break down to release the bioactive agent in the uterus of the patient in response to application of a release agent to the plurality of spheroidal implants.

According to one aspect, this disclosure is directed to a system for treating a gynecological malignancy. The system includes a plurality of implants sufficient in number to substantially fill a uterine cavity of a uterus of a patient to treat the gynecological malignancy. The plurality of implants is configured to release a bioactive agent in the uterus as the plurality of implants break down in the uterus over time.

In embodiments, the plurality of implants may include resorbable material. The resorbable material of the plurality of implants may include carboxy-methyl cellulose.

In embodiments, the bioactive agent may include a chemotherapeutic agent, a hormonal agent, or combinations thereof.

In certain embodiments, each of the plurality of implants may have a spheroidal configuration.

The system may further include a surgical instrument configured to introduce the plurality of implants into the uterus transcervically.

According to yet another aspect, this disclosure is directed to a method of treating a gynecological malignancy. The method includes introducing a plurality of spheroidal implants into a uterus of a patient, and enabling the plurality of spheroidal implants to break down to release a bioactive agent into the uterus to treat the gynecological malignancy.

In aspects, introducing the plurality of spheroidal implants may include introducing the plurality of spheroidal implants into the uterus transcervically.

In aspects, introducing the plurality of spheroidal implants may include advancing the plurality of spheroidal implants through a surgical instrument.

The method may further involve advancing the surgical instrument transvaginally to position the surgical instrument adjacent to a cervix of the patient.

In aspects, enabling the plurality of spheroidal implants to break down to release the bioactive agent may include releasing a chemotherapeutic agent from the plurality of spheroidal implants into the uterus to facilitate treatment of the gynecological malignancy.

In aspects, enabling the plurality of spheroidal implants to break down to release the bioactive agent may include releasing a hormonal agent from the plurality of spheroidal implants into the uterus to facilitate treatment of the gynecological malignancy.

In aspects, introducing the plurality of spheroidal implants into the uterus of the patient may include substantially filling a uterine cavity with the plurality of spheroidal implants.

In aspects, enabling the plurality of spheroidal implants to break down to release a bioactive agent into the uterus to treat the gynecological malignancy may include treating endometrial cancer, endometrial hyperplasia, endometriosis, or combinations thereof.

The details of one or more aspects of this disclosure are set forth in the accompanying drawings and the description below. Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate embodiments of the disclosure and, together with the detailed description of the embodiments given below, serve to explain the principles of the disclosure.

FIG. 1 illustrates portions of female reproductive anatomy; and

FIGS. 2-4 are progressive views illustrating transcervical treatment of the female reproductive anatomy of FIG. 1 with implants including one or more bioactive agents in accordance with the principles of this disclosure.

### DETAILED DESCRIPTION

Aspects of this disclosure are described in detail with reference to the drawings, in which like reference numerals designate identical or corresponding elements in each of the several views. As commonly known, the term “clinician” refers to a doctor (e.g., a surgeon), a nurse, or any other care provider and may include support personnel.

As used herein, the term “biodegradable” in reference to a material shall refer to the property of the material being able to be harmlessly absorbed by the body. In the present application, the terms “biodegradable,” “bioresorbable,” “bioerodable,” and “bioabsorbable” are used interchangeably and are intended to mean the characteristic according to which a material decomposes, or loses structural integrity under body conditions (e.g., enzymatic degradation or hydrolysis) or are broken down (physically or chemically) under physiologic conditions in the body, such that the degradation products are excretable or absorbable by the body after a given period of time. The time period may vary, from about one hour to about several months or more, depending on the chemical nature of the material. In embodiments, the material may not be completely absorbed, provided the non-absorbed material poses no health risks and is biocompatible.

Further, the term “bioactive agent” includes “active therapeutic agent” (ATA) and can be used interchangeably. In its broadest sense, the term “bioactive agent” includes any substance or mixture of substances that have clinical use. The bioactive agents may invoke a biological action, exert a biological effect, or play a role in one or more biological processes. Consequently, bioactive agents may or may not have pharmacological activity per se, e.g., a dye, or fragrance. Alternatively a bioactive agent could be any agent that provides a therapeutic or prophylactic effect, a compound that affects or participates in tissue growth, cell growth, cell differentiation, an anti-adhesive compound, a compound that may be able to invoke a biological action such as an immune response, or could play any other role in one or more biological processes. The bioactive agent may be applied to the disclosed structure in any suitable form of matter, e.g., films, powders, liquids, gels and the like. The type and amount of bioactive agent(s) used will depend, among other factors, on the particular site and condition to be treated.

Examples of classes of bioactive agents which may be utilized in accordance with the present disclosure include anti-adhesives, antimicrobials, analgesics, antipyretics, anesthetics, antiepileptics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, gastrointestinal drugs, diuretics, steroids, lipids, lipopolysaccharides, polysaccharides, platelet activating drugs, clotting factors and enzymes. It is also intended that combinations of bioactive agents may be used.

Anti-adhesive agents can be used to prevent adhesions from forming between the disclosed implants and the surrounding tissues opposite the target tissue. In addition, anti-adhesive agents may be used to prevent adhesions from forming between the implants and packaging material thereof. Some examples of these agents include, but are not limited to hydrophilic polymers such as poly(vinyl pyrrolidone), carboxymethyl cellulose, hyaluronic acid, polyethylene oxide, poly vinyl alcohols, and combinations thereof.

Suitable antimicrobial agents include triclosan, also known as 2,4,4′-trichloro-2′-hydroxydiphenyl ether, chlorhexidine and its salts, including chlorhexidine acetate, chlorhexidine gluconate, chlorhexidine hydrochloride, and chlorhexidine sulfate, silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine, polymyxin, tetracycline, aminoglycosides, such as tobramycin and gentamicin, rifampicin, bacitracin, neomycin, chloramphenicol, miconazole, quinolones such as oxolinic acid, norfloxacin, nalidixic acid, pefloxacin, enoxacin and ciprofloxacin, penicillins such as oxacillin and pipracil, nonoxynol 9, fusidic acid, cephalosporins, and combinations thereof. In addition, antimicrobial proteins and peptides such as bovine lactoferrin and lactoferricin B may be included as a bioactive agent in a bioactive coating of this disclosure.

Other bioactive agents include: local anesthetics; non-steroidal antifertility agents; parasympathomimetic agents; psychotherapeutic agents; tranquilizers; decongestants; sedative hypnotics; steroids; sulfonamides; sympathomimetic agents; vaccines; vitamins, such as vitamin A, B-12, C, D, combinations thereof, and the like; antimalarials; anti-migraine agents; anti-parkinson agents such as L-dopa; anti-spasmodics; anticholinergic agents (e.g., oxybutynin); antitussives; bronchodilators; cardiovascular agents such as coronary vasodilators and nitroglycerin; alkaloids; analgesics; narcotics such as codeine, dihydrocodeinone, meperidine, morphine and the like; non-narcotics such as salicylates, aspirin, acetaminophen, d-propoxyphene and the like; opioid receptor antagonists, such as naltrexone and naloxone; anti-cancer agents; anti-convulsants; anti-emetics; antihistamines; anti-inflammatory agents such as hormonal agents, hydrocortisone, prednisolone, prednisone, non-hormonal agents, allopurinol, indomethacin, phenylbutazone and the like; prostaglandins and cytotoxic drugs; chemotherapeutics, estrogens; antibacterials; antibiotics; anti-fungals; anti-virals; anticoagulants; anticonvulsants; antidepressants; antihistamines; and immunological agents.

Other examples of suitable bioactive agents also include biologics and protein therapeutics, such as, viruses, bacteria, lipids, amino acids, cells, peptides, polypeptides and proteins, analogs, muteins, and active fragments thereof, such as immunoglobulins, antibodies, cytokines (e.g., lymphokines, monokines, chemokines), blood clotting factors, hemopoietic factors, interleukins (IL-2, IL-3, IL-4, IL-6), interferons (β-IFN, α-IFN, and γ-IFN), erythropoietin, nucleases, tumor necrosis factor, colony stimulating factors (e.g., GCSF, GM-CSF, MCSF), insulin, anti-tumor agents and tumor suppressors, blood proteins, fibrin, thrombin, fibrinogen, synthetic thrombin, synthetic fibrin, synthetic fibrinogen, gonadotropins (e.g., FSH, LH, CG, etc.), hormones and hormone analogs (e.g., growth hormone), vaccines (e.g., tumoral, bacterial and viral antigens); somatostatin; antigens; blood coagulation factors; growth factors (e.g., nerve growth factor, insulin-like growth factor); bone morphogenic proteins, TGF-B, protein inhibitors, protein antagonists, and protein agonists; nucleic acids, such as antisense molecules, DNA, RNA, RNAi; oligonucleotides; polynucleotides; and ribozymes.

In the following description, well-known functions or constructions are not described in detail to avoid obscuring this disclosure in unnecessary detail.

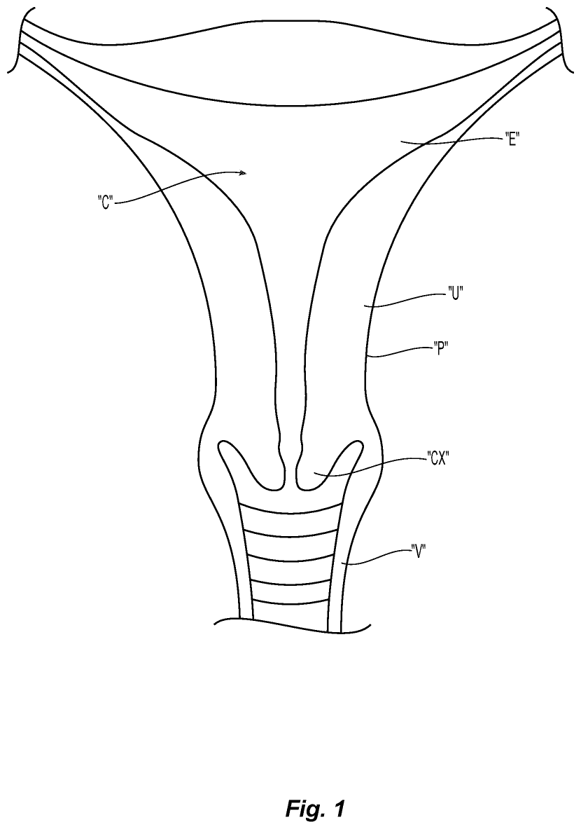

With regard to FIG. 1, female reproductive anatomy of a female patient “P” generally includes a vagina “V” that connects to a uterus “U” defining a uterine cavity “C.” A cervix “Cx” is disposed between the vagina “V” and the uterus “U.” The uterus “U” is lined by an endometrium “E” that can be subject to a gynecologic malignancy such as endometrial cancer, endometrial hyperplasia, and/or endometriosis.

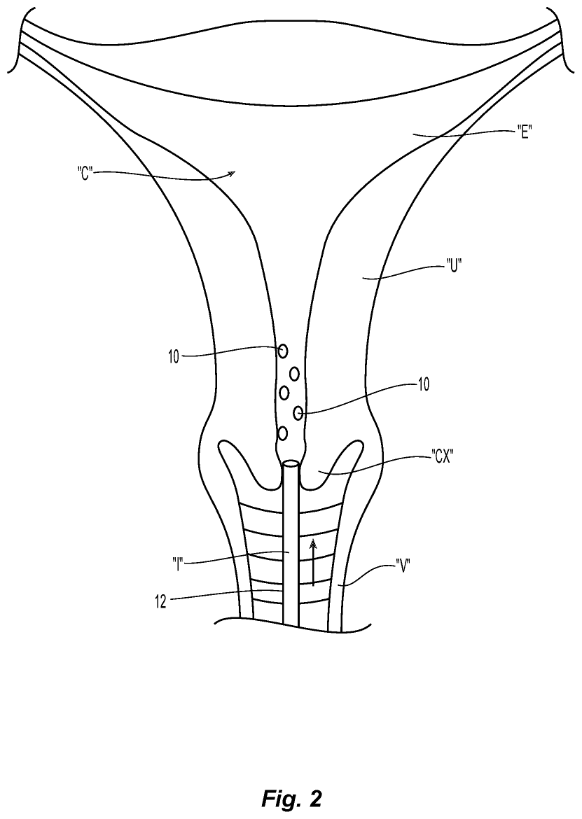

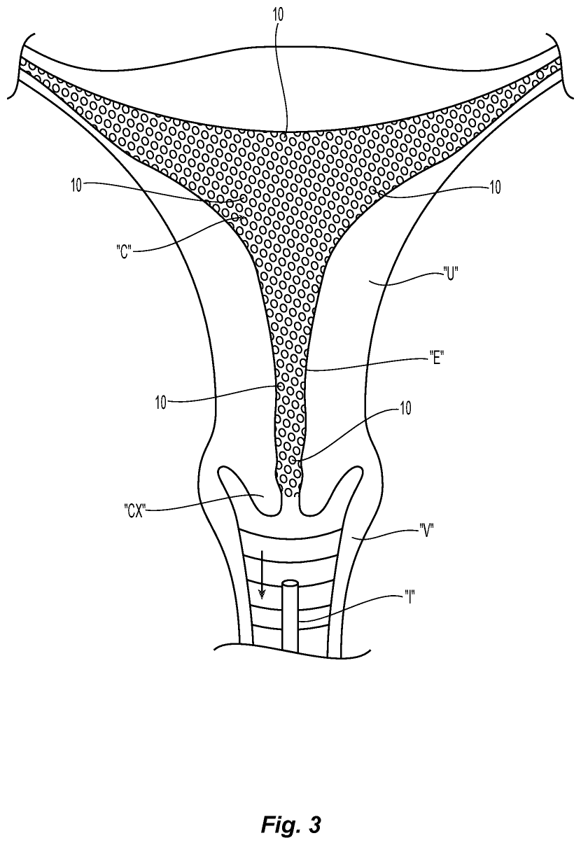

Turning to FIGS. 2-4, in order to treat such gynecologic malignancy, a plurality of implants 10 can be introduced into the uterine cavity “C,” for example, transcervically and/or transvaginally with a surgical instrument 12 configured to dispense the implants 10 into the uterus “U.” Implants 10 are illustrated in FIGS. 2 and 3 as being spheres, spherical, and/or spheroidal, but may include any suitable shape and/or configuration (e.g., circular, non-circular cross-sections, etc.). For example, implants 10 may include, or be made from, dissolving or resorbable material such as carboxy-methyl cellulose or CMC. In embodiments, implants 10 can include one or more bioactive agents “A” such a chemotherapeutic or hormonal agent. In some embodiments, application of one or more bioactive agents “A” (e.g., an exogenous release agent) to implants 10 may facilitate a breakdown of implant 10 to enable release of one or more other bioactive agents “A” supported by implant 10.

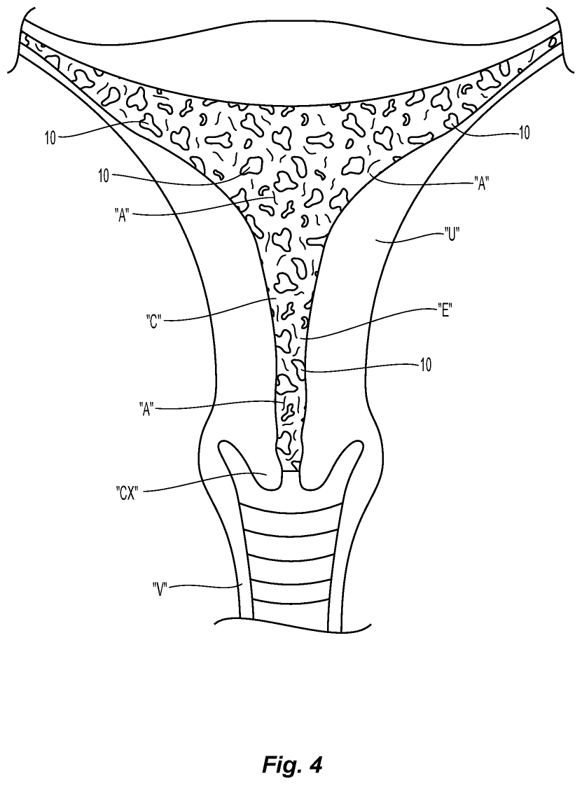

Implants 10 can be introduced one-by-one, or a plurality at a time, via surgical instrument 12 into uterine cavity “C” of the patient “P” until the cavity “C” is filled with implants 10 in abutting relationship with one another (e.g., such as in a bag of marbles) within cavity “C.” Surgical instrument 12 can then be withdrawn, for instance, transvaginally. Over time (e.g., minutes, hours, days, weeks, months, etc.), implants 10 are configured to dissolve, break down and/or resorb into the patient's body so that agent “A” can be released into cavity “C,” as illustrated in FIG. 4, for treating a gynecological malignancy of the patient “P.”

In some embodiments, one or more of these bioactive agents “A” are layered on implant 10. In certain embodiments, one or more of these bioactive agents are impregnated within implant 10.

In embodiments, any of implants 10 can be large enough to prevent one or more of implants 10 from passing through a tubal ostia.

In certain embodiments, one or more implants may be non-active implants (not shown) that may be positioned in the tubal ostia, for example. Such non-active implants may be non-absorbable and may be positioned for selective removal after a predetermined time period. Such non-active implants may be resorbable and may be configured to break down slower than the implants 10 for filling the cavity (e.g., with bioactive agents “A”). These non-active implants can be large enough to block off the fallopian tubes before injecting implants 10 for filing the uterine cavity. In aspects, after the uterine cavity is filled, another non-active implant can be positioned into the cervical canal to maintain implants 10 within the cavity filled while the active implants 10 dissolve.

In some embodiments, the non-active implants can be configured to swell or expand to a larger size after being inserted into the uterus (e.g., through fluid absorption, shape memory material, inflation, etc.).

The various implants disclosed herein may also be configured to be delivered by robotic surgical systems, which may include surgical instrument 12, and what is commonly referred to as “Telesurgery.” Such systems employ various robotic elements to assist the clinician and allow remote operation (or partial remote operation) of surgical instrumentation. Various robotic arms, gears, cams, pulleys, electric and mechanical motors, etc. may be employed for this purpose and may be designed with a robotic surgical system to assist the clinician during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients. For a detailed description of exemplary medical work stations and/or components thereof, reference may be made to U.S. Pat. No. 8,828,023, and PCT Application Publication No. WO2016/025132, the entire contents of each of which are incorporated by reference herein.

Persons skilled in the art will understand that the structures and methods specifically described herein and illustrated in the accompanying figures are non-limiting exemplary embodiments, and that the description, disclosure, and figures should be construed merely as exemplary of particular embodiments. It is to be understood, therefore, that this disclosure is not limited to the precise embodiments described, and that various other changes and modifications may be effected by one skilled in the art without departing from the scope or spirit of this disclosure. Additionally, it is envisioned that the elements and features illustrated or described in connection with one exemplary embodiment may be combined with the elements and features of another without departing from the scope of this disclosure, and that such modifications and variations are also intended to be included within the scope of this disclosure. Indeed, any combination of any of the disclosed elements and features is within the scope of this disclosure. Accordingly, the subject matter of this disclosure is not to be limited by what has been particularly shown and described.

What is claimed is:

1. A spheroidal implant system, comprising
a plurality of spheroidal implants configured to be positioned together within a uterus of a patient, each spheroidal implant including a bioactive agent that is selectively releasable into the uterus to enable the spheroidal implants to cooperate together to simultaneously treat a gynecological malignancy of the patient.

2. The spheroidal implant system of claim 1, wherein the bioactive agent includes a chemotherapeutic agent.

3. The spheroidal implant system of claim 1, wherein the bioactive agent includes a hormonal agent.

4. The spheroidal implant system of claim 1, wherein each of the plurality of spheroidal implants includes a resorbable material.

5. The spheroidal implant system of claim 4, wherein the resorbable material includes carboxy-methyl cellulose.

6. The spheroidal implant system of claim 4, wherein the resorbable material is configured to break down to release the bioactive agent in the uterus of the patient in response to application of a release agent to the plurality of spheroidal implants.

7. A system for treating a gynecological malignancy, the system comprising:
a plurality of implants sufficient in number to substantially fill a uterine cavity of a uterus of a patient to treat the gynecological malignancy, the plurality of implants configured to release a bioactive agent in the uterus as the plurality of implants break down in the uterus over time.

8. The system of claim 7, wherein the plurality of implants includes resorbable material.

9. The system of claim 8, wherein the resorbable material includes carboxy-methyl cellulose.

10. The system of claim 7, wherein the bioactive agent includes a chemotherapeutic agent, a hormonal agent, or combinations thereof.

11. The system of claim 7, wherein each of the plurality of implants has a spheroidal configuration.

12. The system of claim 7, further comprising a surgical instrument, the surgical instrument configured to introduce the plurality of implants into the uterus transcervically.

13. A method of treating a gynecological malignancy, the method comprising:
introducing a plurality of spheroidal implants into a uterus of a patient; and
enabling the plurality of spheroidal implants to break down to release a bioactive agent into the uterus to treat the gynecological malignancy.

14. The method of claim 13, wherein introducing the plurality of spheroidal implants includes introducing the plurality of spheroidal implants into the uterus transcervically.

15. The method of claim 14, wherein introducing the plurality of spheroidal implants includes advancing the plurality of spheroidal implants through a surgical instrument.

16. The method of claim 15, further comprising advancing the surgical instrument transvaginally to position the surgical instrument adjacent to a cervix of the patient.

17. The method of claim 13, wherein enabling the plurality of spheroidal implants to break down to release the bioactive agent includes releasing a chemotherapeutic agent from the plurality of spheroidal implants into the uterus to facilitate treatment of the gynecological malignancy.

18. The method of claim 13, wherein enabling the plurality of spheroidal implants to break down to release the bioactive agent includes releasing a hormonal agent from the plurality of spheroidal implants into the uterus to facilitate treatment of the gynecological malignancy.

19. The method of claim 13, wherein introducing the plurality of spheroidal implants into the uterus of the patient includes substantially filling a uterine cavity with the plurality of spheroidal implants.

20. The method of claim 13, wherein enabling the plurality of spheroidal implants to break down to release a bioactive agent into the uterus to treat the gynecological malignancy includes treating endometrial cancer, endometrial hyperplasia, endometriosis, or combinations thereof.
